SEQUENCE LISTING (12 sequences)

SEQ ID NO: 1 - DNA, 20 bp, Rattus rattus
ctctcaccctattaaccact

SEQ ID NO: 2 - DNA, 20 bp, Rattus rattus
gttaaaagtgcataccgcca

SEQ ID NO: 3 - DNA, 26 bp, Rattus rattus
gcttatgataatctcaacaaagttcg

SEQ ID NO: 4 - DNA, 20 bp, Rattus rattus
atgttctcatgtctgaagcg

SEQ ID NO: 5 - DNA, 26 bp, Mus musculus
gccagcctgacccatagccataatat

SEQ ID NO: 6 - DNA, 25 bp, Mus musculus
gagagattttatgggtgtaatgcgg

SEQ ID NO: 7 - DNA, 24 bp, Mus musculus
cccagctactaccatcattcaagt

SEQ ID NO: 8 - DNA, 25 bp, Mus musculus
gatggtttgggagattggttgatgt

SEQ ID NO: 9 - DNA, 26 bp, Rattus rattus
aaaatccccgcaaacaatgaccaccc

SEQ ID NO: 10 - DNA, 26 bp, Rattus rattus
ggcaattaagagtgggatggagccaa

SEQ ID NO: 11 - DNA, 27 bp, Rattus rattus
cctcccattcattatcgccgcccttgc

SEQ ID NO: 12 - DNA, 27 bp, Rattus rattus
gtctgggtctcctagtaggtctgggaa

(19) United States
(12) Patent Application Publication
(10) Pub. No.: US 2021/0177861 A1
(43) Pub. Date: Jun. 17, 2021

(54) NRF2 ACTIVATOR FOR THE TREATMENT OF ACUTE LUNG INJURY, ACUTE RESPIRATORY DISTRESS SYNDROME AND MULTIPLE ORGAN DYSFUNCTION SYNDROME

(71) Applicant: GLAXOSMITHKLINE INTELLECTUAL PROPERTY DEVELOPMENT LIMITED, Brentford, Middlesex (GB)

(72) Inventor: William RUMSEY, Collegeville, PA (US)

(21) Appl. No.: 16/771,062

(22) PCT Filed: Dec. 11, 2018
(86) PCT No.: PCT/IB2018/059884

§ 371 Date: Jun. 9, 2020

Related U.S. Application Data
(60) Provisional application No. 62/597,131, filed on Dec. 11, 2017.

(51) Int. Cl. A61K 31/554; A61P 11/00

(52) U.S. Cl. CPC A61K 31/554; A61P 11/00

(57) ABSTRACT

The present invention relates to the use of an NRF2 activator to treat respiratory diseases. In particular, the present invention relates to the treatment of respiratory diseases, in a mammal, in which related organ failure accompanied by accumulation of alveolar fluid, hypoxemia, cough, wheezing, dyspnea, hyperpnea and pulmonary inflammation has occurred.

### FIELD OF INVENTION

The present invention relates to the use of an NRF2 activator to treat respiratory diseases. In particular, the present invention relates to the treatment of respiratory diseases, in a mammal, in which related organ failure accompanied by accumulation of alveolar fluid, hypoxemia, cough, wheezing, dyspnea, hyperpnea and pulmonary inflammation has occurred.

### SEQUENCE LISTING

The instant application contains a Sequence Listing which has been filed electronically in ASCII format and is hereby incorporated by reference in its entirety. Said ASCII copy, created on 10 Dec. 2018, is named PR66506.txt and is 2,004 bytes in size.

### BACKGROUND OF THE INVENTION

Acute lung injury (ALI) or its more severe form, acute respiratory distress syndrome (ARDs) results from a seemingly diverse array of etiologies such as bacterial infection, inhalation of toxic substances, direct injury to the lung, sepsis, burn, substantial levels of blood transfusion, eclampsia, etc. Most frequently, ALI and ARDs occur in hospital settings where 5-10% of patients are within intensive care units (ICU) and up to 25% of ventilated individuals become afflicted by this syndrome (Cannon et al., Crit Care Clin 33: 259-275, 2017; Umbrello et al., Int J Mol Sci 18: 64-84, 2017). It is a heterogenous condition that results from a diverse ICU population. Although the incidence is declining, it is often underdiagnosed, and mortality remains high from 20-40%. The mechanisms underpinning the cause of death are not clearly delineated, however in severe ARDs the resulting oxygen debt is thought to be a contributing factor. Endothelial and epithelial cell injury, with consequent enhancement of vascular permeability and inflammation are hallmark features of the condition. Moreover, the rupture of epithelial integrity, specifically the type II alveolar cells that are responsible for removal of fluid from the alveolar space and for surfactant production may promote the progression of atelectasis and loss of gas exchange. Often, ALI and ARDs contain a systemic component, in particular when coupled with infection of the lung. Many patients succumb to multiple organ dysfunction syndrome (MODS) rather than overt respiratory failure and those that survive may suffer from neurocognitive decline, and decreased quality of life, indicating crosstalk between the lung and other organs (Quillez et al., Curr Opin Crit Care. 18(1):23-8, 2012).

A loss of mitochondrial (mt) function has been a central component in the pathogenesis of ALI, ARDs and MODS. Recent studies have found that fragments of mtDNA, so-called mtDAMPs, are released into the circulation following severe injury which can serve as mediators of inflammation in areas distal to the site of insult (Zhang et al., Nature 464: 104-107, 2010). MtDNA is thought to be more susceptible to damage and mutation than nuclear DNA. The lack of co-existent histone complexes; the single stranded nature of mtDNA replication; and its physical proximity to the primary source of endogenous reactive oxygen species (ROS), i.e., the respiratory chain, render mtDNA vulnerable to lesion formation and mutation. Oxidative stress induces the degradation of mtDNA which is accompanied by the reduction of mitochondrial energy production and cell viability (Shokolenko et al., 2009, Nuc Acids Res 37:8, 2539-2548; Shokolenko et al., 2013, DNA Repair 12:7, 488-499). The loss of mtDNA integrity promotes mitochondrial fragmentation (Shokolenko ibid) and expulsion of the DNA from the cell although this mechanism is yet to be defined. Patients who have developed MODS have higher levels of plasma mtDAMPS and those with amounts above the median level have a greater risk of mortality (Simmon et al., Ann Surg 258 (4): 591-598, 2013). In patients requiring massive transfusion of blood who then developed ARDs, there were increased levels of mtDAMPS in the transfusion products (Simmons et al., J Trauma Acute Care Surg, 2017). Exposure of a human endothelial monolayer to purified mtDNA results in a leaky, compromised barrier that arises from neutrophil dependent and independent mechanisms (Sun et al., PLoS One. 2013; 8(3):e59989. doi: 10.1371). The direct administration of mtDAMPS to isolated lung preparations or to animals promotes ALI and multiple organ failure (Kuck et al., Am J Physiol Lung Cell Mol Physiol 308(10: L1078-L1086, 2015, Zhang et al., Int J Mol Sci 17: 142514-41, 2016). Of note, only mtDNA and not nuclear DNA resulted in ALI and systemic inflammation (Zhang et al., ibid). MtDNA contain un-methylated cytosine phosphate guanine motifs, CPGs, which stimulate the immune system most likely through interaction with the TLR9 receptor (Zhang, et al., ibid).

In a murine model of S. aureus-induced pneumonia and consequent ALI, the NRF2 (Nfe212) transcription factor is activated, primarily in alveolar type II cells, to promote mitochondrial biogenesis and counter inflammation (Athale et al., Free Radic Biol Med 53(8): 1584-1594, 2012). By contrast, the molecular deletion of this transcription factor suppresses mitogenesis and enhances inflammation, thereby exacerbating ALI. Genetic variation of NRF2 provide susceptibility to ALI in both mice and in humans (Marzec et al., FASEB J 21: 2237-2246, 2007, Cho et al., Antioxidants Redox Signaling 22:/4; 325338, 2015). Moreover, the treatment of animals with Bardoxolone, an NRF2 activator, protected them from hyperoxia-induced ALI (Reddy et al., Am J Respir Crit Care Med 180: 867-874, 2009). These data provide both genetics and pharmacological linkage of the NRF2 pathway to ALI.

The use of the herbicide, paraquat (PQ: 1,1′-dimethyl-4,4′-bipyridinium dichloride) is currently forbidden in the United States and Europe but remains a widely-used agent in developing countries. When sprayed in fields, PQ is inhaled by workers or can contact their skin and presents a potentially lethal toxicological challenge to humans (Smith and Heath J Clin Pathol Suppl (R Coll Pathol). 9:81-93,1975). PQ is a redox cycler that associates with the mitochondrial respiratory chain, principally at Complex I where it converts molecular oxygen to the superoxide radical which damages mitochondrial lipids, proteins, and DNA (Cochemé and Murphy J Biol Chem. 283(4):1786-1798, 2008). In the lung, the principal cellular target of PQ's destructive action is the alveolar epithelium, specifically Type I and II pneumocytes (Smith and Heath J Clin Pathol Suppl (R Coll Pathol). 9:81-93,1975). A single intraperitoneal administration of the agent to rats results in rapid swelling of Type I alveolar epithelium with additional degenerative changes in Type II cells (ibid). Progressive damage, i.e., sloughing of the epithelium, alveolar edema, congested capillaries and inflammation with mononuclear cells apparent in the alveolar spaces can be found within a few days. In a murine model of PQ-induced ALI, the levels of mtDNA were increased in the systemic circulation and bronchoalveolar lavage fluid (Li et al., Biomed Res Inter 2015 Art ID 386952). Protection from PQ-induced lung injury and survival was afforded by treatment with DNasel, presumably targeting expulsed mtDNA.

Ozone is the most prevalent form of air pollution and the most dangerous causing premature death due to respiratory diseases (Jerrett et al., N Engl J Med. 360(11):1085-95, 2009). Even low levels of ozone exposure to humans is associated with ALI/ARDS in at risk critically-ill persons (Ware et al., Am J Respir Crit Care Med.; 193(10):1143-50, 2016). Ozone, and other environmental hazards like tobacco smoke, may serve as risks factors for the development of ALI/ARDs. Similar to PQ, ozone invokes oxidative stress within the cells that they contact and adversely affect mitochondrial function.

ALI and ARDS remains a global health problem for which there are few medical recourses or medications. In general, ALI/ARDs presents in afflicted persons as hypoxemia with bilateral pulmonary infiltrates. The pulmonary edema is of non-cardiogenic origin and the compliance of the lung is adversely affected. The small vessels of the pulmonary circulation become leaky permitting passage of fluid and proteins into the gas exchange units or alveoli thereby compromising the diffusion of oxygen and the removal of carbon dioxide to and from the blood stream. Treatment is largely dependent upon mechanical maneuvers to improve the ventilation:perfusion ratio of the lung. Pharmacological treatments are few with bronchodilators, neuromuscular blockade and corticosteroids demonstrating mixed results.

Thus, there is a clear unmet medical need for therapy, preferably in the form of a suitable small molecule which will treat respiratory diseases in a mammal, in which related organ failure accompanied by accumulation of alveolar fluid, hypoxemia and inflammation has occurred. The current application teaches the novel finding that an NRF2 activator (S)-3-(3-(((R)-4-ethyl-1,1-dioxido-3,4-dihydro-2H-pyrido[2,3-b][1,4,5]oxathiazepin-2-yl)methyl)-4-methylphenyl)-3-(1-ethyl-4-methyl-1H-benzo[d][1,2,3]triazol-5-yl)propanoic acid (Compound I) or a pharmaceutically acceptable salt thereof, is effective in preventing the loss of pulmonary endothelial barrier function as evidenced by the maintenance of the lung wet:dry ratio that leads, in part, to ALI. Moreover, the blockade of mtDNA damage provides a mechanistic link to these protective effects and others which also include a reduced level of pulmonary inflammation, i.e., decreased numbers of immune cells in the bronchoalveolar lavage fluid.

### SUMMARY OF THE INVENTION

In one aspect, the present invention is directed to the novel use of an NRF2 activator, or a pharmaceutically acceptable salt thereof, for the treatment of acute lung injury. In one embodiment, the NRF2 activator is Compound I or a pharmaceutically acceptable salt thereof.

In another aspect, the present invention is directed to the novel use of an NRF2 activator, or a pharmaceutically acceptable salt thereof, for the treatment of acute respiratory distress syndrome. In one embodiment, the NRF2 activator is Compound I or a pharmaceutically acceptable salt thereof.

In yet another aspect, the present invention is directed to the novel use of an NRF2 activator, or a pharmaceutically acceptable salt thereof, for the treatment of multiple organ dysfunction syndrome. In one embodiment, the NRF2 activator is Compound I or a pharmaceutically acceptable salt thereof.

In still another aspect, the present invention is directed to a method of treating acute lung injury in a mammal in need thereof, comprising administering an effective amount of an NRF2 activator. In one embodiment, the NRF2 activator is Compound I or a pharmaceutically acceptable salt thereof.

In another aspect, the present invention is directed to a method of treating acute respiratory distress syndrome in a mammal in need thereof, comprising administering an effective amount of an NRF2 activator. In one embodiment, the NRF2 activator is Compound I or a pharmaceutically acceptable salt thereof.

In another aspect, the present invention is directed to a method of treating multiple organ dysfunction syndrome in a mammal in need thereof, comprising administering an effective amount of an NRF2 activator. In one embodiment, the NRF2 activator is Compound I or a pharmaceutically acceptable salt thereof.

In one aspect, the present invention is directed to a method of treating the symptoms of acute lung injury in a mammal in need thereof, comprising administering an effective amount of an NRF2 activator. The symptoms include but are not limited to, an accumulation of alveolar fluid, hypoxemia, cough, wheezing, dyspnea, hyperpnea and pulmonary inflammation. Suitably, on a cellular level, these symptoms are exhibited by increased neutrophil and macrophage accumulation in the bronchoalveolar lavage fluid. In one embodiment, the NRF2 activator is Compound I or a pharmaceutically acceptable salt thereof.

In a further aspect, the present invention is directed to the use of an NRF2 activator, or a pharmaceutically acceptable salt thereof in the manufacture of a medicament for the treatment of acute lung injury. In one embodiment, the NRF2 activator is Compound I or a pharmaceutically acceptable salt thereof.

In still a further aspect, the present invention is directed to the use of an NRF2 activator, or a pharmaceutically acceptable salt thereof in the manufacture of a medicament for the treatment of acute respiratory distress syndrome. In one embodiment, the NRF2 activator is Compound I or a pharmaceutically acceptable salt thereof.

In still yet a further aspect, the present invention is directed to the use of an NRF2 activator, or a pharmaceutically acceptable salt thereof in the manufacture of a medicament for the treatment of multiple organ dysfunction syndrome. In one embodiment, the NRF2 activator is Compound I or a pharmaceutically acceptable salt thereof.

In another aspect, the present invention is directed to the use of an NRF2 activator, or a pharmaceutically acceptable salt thereof in the manufacture of a medicament for the treatment of the symptoms of acute lung injury including, but not limited to an accumulation of alveolar fluid, hypoxemia, cough, wheezing, dyspnea, hyperpnea and pulmonary inflammation. Suitably, on a cellular level, these symptoms are exhibited by increased neutrophil and macrophage accumulation in the bronchoalveolar lavage fluid. In one embodiment, the NRF2 activator is Compound I or a pharmaceutically acceptable salt thereof.

In yet another aspect, the present invention is directed to an NRF2 activator or a pharmaceutically acceptable salt thereof, for use in the treatment of acute lung injury. In one embodiment, the NRF2 activator is Compound I or a pharmaceutically acceptable salt thereof.

In yet another aspect, the present invention is directed to an NRF2 activator or a pharmaceutically acceptable salt thereof, for use in the treatment of acute respiratory distress syndrome. In one embodiment, the NRF2 activator is Compound I or a pharmaceutically acceptable salt thereof.

In yet another aspect, the present invention is directed to an NRF2 activator or a pharmaceutically acceptable salt thereof, for use in the treatment of multiple organ dysfunction syndrome. In one embodiment, the NRF2 activator is Compound I or a pharmaceutically acceptable salt thereof.

In yet another aspect, the present invention is directed to an NRF2 activator or a pharmaceutically acceptable salt thereof, for use in the treatment of the symptoms of acute lung injury, including, but not limited to, an accumulation of alveolar fluid, hypoxemia, cough, wheezing, dyspnea, hyperpnea and pulmonary inflammation. Suitably, on a cellular level, these symptoms are exhibited by increased neutrophil and macrophage accumulation in the bronchoalveolar lavage fluid. In one embodiment, the NRF2 activator is Compound I or a pharmaceutically acceptable salt thereof.

It will be understood that for any of the methods of treatment or uses discussed above, in one embodiment, the NRF2 activator is the free acid Compound I.

### BRIEF DESCRIPTION OF THE FIGURES

FIG. 1 depicts the effect of NRF2 activator Compound I on PQ-induced changes of lung function and pulmonary edema formation. All data represent mean±S.E.M (N=5). FIG. 1A provides time-dependent changes in lung function, expressed as Penh. FIG. 1B shows changes in lung wet:dry weight ratio in response to PQ and PQ plus Compound I. Compound I was administered as a suspension by intra-tracheal delivery 24 hrs. prior to PQ (0.05 mg/kg i.t.) administration.

FIG. 2 depicts the effect of NRF2 activator Compound I on measurements of pulmonary inflammation. These data show the reduction in inflammatory immune cells in bronchoalveolar lavage fluid obtained from paraquat-treated rats. All data represent means±S.E.M.

FIG. 3 depicts the effect of NRF2 activator Compound I on relative mtDNA copy number, a measure of mtDNA damage (FIG. 3A), NRF2-mediated gene expression (FIG. 3B) and 8-OHdG levels (FIG. 3C). Other genes (GCLC, HO-1, etc., exception being TXNRD1) were also up-regulated relative to the PQ treated vehicle control animals. All data represent means±S.E.M Asterisks *, **, *** refer to p<0.05, 0.01, 0.001, respectively.

FIG. 4 depicts the effect of NRF2 activator Compound I on ozone-induced changes in lung wet to dry weight ratio (FIG. 4A) and relative mtDNA copy number (FIG. 4B). Rats were administered the NRF2 activator (3 μmol/kg i.t.) 24 hours prior to ozone exposure (1 ppm of ozone for 3 hours). Four hrs later the animals were sacrificed. All data represent means±S.E.M. (*, **, *** refer to p<0.05, 0.01 and 0.001, respectively).

FIG. 5 depicts the protection offered by NRF2 activator Compound I against ozone-induced death (FIG. 5A) and loss of glutathione (FIG. 5B). Due to an unknown faulty ventilation system, ozone in the chamber built up to levels (unable to quantify) above those normally used in other studies. The intended exposure was 1 ppm. For those animals that survived, tissue values of NRF2-related parameters were examined 24 hrs. after ozone exposure. In addition, animals exposed to ozone within the control group were combined. All data represent means±S.E.M. (*, **, *** refer to p<0.05, 0.01 and 0.001, respectively).

FIG. 6 depicts the protective effect of administering NRF2 Compound I on the degradation or breakdown of the alveolar barrier in mice. Mice were exposed to ozone (1.5 ppm) for 3 hrs, twice per week for 3 weeks. Compound 1 was administered for 5 days/week, with the first dose administered 1 hour prior to first ozone administration. Blood/serum was collected 2 hrs after the final ozone exposure. Surfactant protein-D was measured using a commercially available ELISA kit. All data represent mean+/−S.E.M.

### DETAILED DESCRIPTION OF THE INVENTION

The NRF2 activator (S)-3-(3-(((R)-4-ethyl-1,1-dioxido-3,4-dihydro-2H-pyrido[2,3-][1,4,5]oxathiazepin-2-yl)methyl)-4-methylphenyl)-3-(1-ethyl-4-methyl-1H-benzo[d][1,2,3]triazol-5-yl)propanoic acid (Compound I), or a pharmaceutically acceptable salt thereof, is described in PCT application WO 2015/092713A1, published on Jun. 25, 2015, incorporated herein by reference. The preparation of the specific NRF2 activator claimed herein is found in Example 146 and has the following structure:

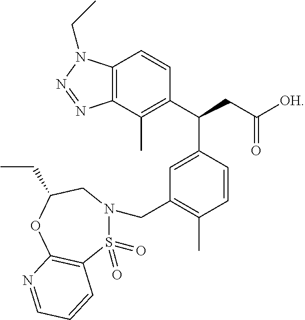 [SMILES: CC[C@@H]1CN(Cc2cc([C@H](CC(=O)O)c3ccc4c(nnn4CC)c3C)ccc2C)S(=O)(=O)c2cccnc2O1]

As used herein, “pharmaceutically acceptable” refers to those compounds, materials, compositions, and dosage forms which are, within the scope of sound medical judgment, suitable for use in contact with the tissues of human beings and animals without excessive toxicity, irritation, or other problem or complication, commensurate with a reasonable benefit/risk ratio.

The methods of treatment of the invention comprise administering an effective amount of a Compound I or a pharmaceutically-acceptable salt thereof to a mammal in need thereof.

As used herein, “treat” in reference to a condition means: (1) to ameliorate or prevent the condition or one or more of the biological manifestations of the condition, (2) to interfere with (a) one or more points in the biological cascade that leads to or is responsible for the condition or (b) one or more of the biological manifestations of the condition, (3) to alleviate one or more of the symptoms or effects associated with the condition, or (4) to slow the progression of the condition or one or more of the biological manifestations of the condition.

The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such condition or biological manifestation thereof.

As used herein, “effective amount” or “an effective amount” in reference to Compound I, or a pharmaceutically acceptable salt thereof, refers to an amount of the compound sufficient to treat the patient's condition but low enough to avoid serious side effects (at a reasonable benefit/risk ratio) within the scope of sound medical judgment. An effective amount of the compound will vary depending on factors such as the route of administration chosen; the condition being treated; the severity of the condition being treated; the age, size, weight, and physical condition of the patient being treated; the medical history of the patient to be treated; the duration of the treatment; the nature of concurrent therapy; the desired therapeutic effect; and like factors, but can nevertheless be routinely determined by the skilled artisan.

As used herein, “mammal” refers to a human or other animal. It will be understood that the patient to be treated with Compound I, or a pharmaceutically acceptable salt thereof, is a mammal, preferably a human.

Compound I, or a pharmaceutically acceptable salt thereof, may be administered by any suitable route of administration, including systemic administration. Systemic administration includes oral administration, parenteral administration, transdermal administration, rectal administration, and administration by inhalation. Parenteral administration refers to routes of administration other than enteral, transdermal, or by inhalation, and is typically by injection or infusion. Parenteral administration includes intravenous, intramuscular, and subcutaneous injection or infusion. Inhalation refers to administration into the patient's lungs whether inhaled through the mouth or through the nasal passages.

Suitably, Compound I, or a pharmaceutically acceptable salt thereof, is administered via inhalation.

Suitably, Compound I, or a pharmaceutically acceptable salt thereof, is administered parenterally.

In one embodiment, Compound I, or a pharmaceutically acceptable salt thereof, is administered via inhalation.

In one embodiment, the free acid Compound I is administered via inhalation.

Compound I, or a pharmaceutically acceptable salt thereof, may be administered once or according to a dosing regimen wherein a number of doses are administered at varying intervals of time for a given period of time. For example, doses may be administered one, two, three, or four times per day. Doses may be administered until the desired therapeutic effect is achieved or indefinitely to maintain the desired therapeutic effect. Suitable dosing regimens for Compound I, or a pharmaceutically acceptable salt thereof, depend on the pharmacokinetic properties of that compound, such as absorption, distribution, and half-life, which can be determined by the skilled artisan. In addition, suitable dosing regimens, including the duration such regimens are administered, for Compound I, or a pharmaceutically acceptable salt thereof, depend on the condition being treated, the severity of the condition being treated, the age and physical condition of the patient being treated, the medical history of the patient to be treated, the nature of concurrent therapy, the desired therapeutic effect, and like factors within the knowledge and expertise of the skilled artisan. It will be further understood by such skilled artisans that suitable dosing regimens may require adjustment given an individual patient's response to the dosing regimen or over time as individual patient needs change.

Typical daily dosages may vary depending upon the particular route of administration chosen. Typical dosages for oral administration range from 1 mg to 1000 mg per person per day. Preferred dosages are 1-500 mg once daily, more preferred is 1-100 mg per person per day. IV dosages range from 0.1-000 mg/day, preferred is 0.1-500 mg/day, and more preferred is 0.1-100 mg/day. Inhaled daily dosages range from 10 ug-10 mg/day, with preferred 10 ug-2 mg/day, and more preferred 50 ug-500 ug/day.

Additionally, Compound I, or a pharmaceutically acceptable salt thereof, may be administered as a prodrug. As used herein, a “prodrug” of Compound I, or a pharmaceutically acceptable salt thereof, is a functional derivative of the compound which, upon administration to a patient, eventually liberates Compound I, or a pharmaceutically acceptable salt thereof, in vivo. Administration of Compound I, or a pharmaceutically acceptable salt thereof, as a prodrug may enable the skilled artisan to do one or more of the following: (a) modify the onset of the compound in vivo; (b) modify the duration of action of the compound in vivo; (c) modify the transportation or distribution of the compound in vivo; (d) modify the solubility of the compound in vivo; and (e) overcome a side effect or other difficulty encountered with the compound. Typical functional derivatives used to prepare prodrugs include modifications of the compound that are chemically or enzymatically cleaved in vivo. Such modifications, which include the preparation of phosphates, amides, ethers, esters, thioesters, carbonates, and carbamates, are well known to those skilled in the art.

### Compositions

The compounds of the invention will normally, but not necessarily, be formulated into pharmaceutical compositions prior to administration to a patient. Accordingly, in another aspect the invention is directed to pharmaceutical compositions comprising Compound I, or a pharmaceutically acceptable salt thereof, and one or more pharmaceutically-acceptable excipients.

The pharmaceutical compositions of the invention may be prepared and packaged in bulk form wherein a safe and effective amount of Compound I, or a pharmaceutically acceptable salt thereof, can be extracted and then given to the patient such as with powders or syrups. Alternatively, the pharmaceutical compositions of the invention may be prepared and packaged in unit dosage form wherein each physically discrete unit contains a safe and effective amount of Compound I, or a pharmaceutically acceptable salt thereof. When prepared in unit dosage form, the pharmaceutical compositions of the invention typically contain from 1 mg to 1000 mg of the active agent.

The pharmaceutical compositions of the invention typically contain one compound of the invention. However, in certain embodiments, the pharmaceutical compositions of the invention may optionally further comprise one or more additional pharmaceutically active compounds.

As used herein, “pharmaceutically-acceptable excipient” means a pharmaceutically acceptable material, composition or vehicle involved in giving form or consistency to the pharmaceutical composition. Each excipient must be compatible with the other ingredients of the pharmaceutical composition when commingled such that interactions which would substantially reduce the efficacy of Compound I, or a pharmaceutically acceptable salt thereof, when administered to a patient and interactions which would result in pharmaceutical compositions that are not pharmaceutically acceptable are avoided. In addition, each excipient must of course be of sufficiently high purity to render it pharmaceutically-acceptable.

Compound I, or a pharmaceutically acceptable salt thereof, and the pharmaceutically-acceptable excipient or excipients will typically be formulated into a dosage form adapted for administration to the patient by the desired route of administration. For example, dosage forms include those adapted for (1) oral administration such as tablets, capsules, caplets, pills, troches, powders, syrups, elixirs, suspensions, solutions, emulsions, sachets, and cachets; (2) parenteral administration such as sterile solutions, suspensions, and powders for reconstitution; and (3) inhalation such as dry powders, aerosols, suspensions, and solutions.

Suitable pharmaceutically-acceptable excipients will vary depending upon the particular dosage form chosen. In addition, suitable pharmaceutically-acceptable excipients may be chosen for a particular function that they may serve in the composition. For example, certain pharmaceutically-acceptable excipients may be chosen for their ability to facilitate the production of uniform dosage forms. Certain pharmaceutically-acceptable excipients may be chosen for their ability to facilitate the production of stable dosage forms. Certain pharmaceutically-acceptable excipients may be chosen for their ability to facilitate the carrying or transporting of the compound or compounds of the invention once administered to the patient from one organ, or portion of the body, to another organ, or portion of the body. Certain pharmaceutically-acceptable excipients may be chosen for their ability to enhance patient compliance.

Suitable pharmaceutically-acceptable excipients include the following types of excipients: diluents, fillers, binders, disintegrants, lubricants, glidants, granulating agents, coating agents, wetting agents, solvents, co-solvents, suspending agents, emulsifiers, sweeteners, flavoring agents, flavor masking agents, coloring agents, anticaking agents, humectants, chelating agents, plasticizers, viscosity increasing agents, antioxidants, preservatives, stabilizers, surfactants, and buffering agents. The skilled artisan will appreciate that certain pharmaceutically-acceptable excipients may serve more than one function and may serve alternative functions depending on how much of the excipient is present in the formulation and what other ingredients are present in the formulation.

Skilled artisans possess the knowledge and skill in the art to enable them to select suitable pharmaceutically-acceptable excipients in appropriate amounts for use in the invention. In addition, there are a number of resources that are available to the skilled artisan which describe pharmaceutically-acceptable excipients and may be useful in selecting suitable pharmaceutically-acceptable excipients. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press).

The pharmaceutical compositions of the invention are prepared using techniques and methods known to those skilled in the art. Some of the methods commonly used in the art are described in Remington's Pharmaceutical Sciences (Mack Publishing Company).

In one aspect, the invention is directed to a solid oral dosage form such as a tablet or capsule comprising a safe and effective amount of Compound I, or a pharmaceutically acceptable salt thereof, and a diluent or filler. Suitable diluents and fillers include lactose, sucrose, dextrose, mannitol, sorbitol, starch (e.g. corn starch, potato starch, and pre-gelatinized starch), cellulose and its derivatives (e.g. microcrystalline cellulose), calcium sulfate, and dibasic calcium phosphate. The oral solid dosage form may further comprise a binder. Suitable binders include starch (e.g. corn starch, potato starch, and pre-gelatinized starch), gelatin, acacia, sodium alginate, alginic acid, tragacanth, guar gum, povidone, and cellulose and its derivatives (e.g. microcrystalline cellulose). The oral solid dosage form may further comprise a disintegrant. Suitable disintegrants include crospovidone, sodium starch glycolate, croscarmellose, alginic acid, and sodium carboxymethyl cellulose. The oral solid dosage form may further comprise a lubricant. Suitable lubricants include stearic acid, magnesium stearate, calcium stearate, and talc.

In another aspect, the invention is directed to a dosage form adapted for administration to a patient parenterally including subcutaneous, intramuscular, intravenous or intradermal. Pharmaceutical formulations adapted for parenteral administration include aqueous and non-aqueous sterile injection solutions which may contain anti-oxidants, buffers, bacteriostats, and solutes that render the formulation isotonic with the blood of the intended recipient; and aqueous and non-aqueous sterile suspensions which may include suspending agents and thickening agents. The formulations may be presented in unit-dose or multi-dose containers, for example sealed ampules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules, and tablets.

In another aspect, the invention is directed to a dosage form adapted for administration to a patient by inhalation. For example, Compound I, or a pharmaceutically acceptable salt thereof, may be inhaled into the lungs as a dry powder, an aerosol, a suspension, or a solution.

Dry powder compositions for delivery to the lung by inhalation typically comprise Compound I, or a pharmaceutically acceptable salt thereof, as a finely divided powder together with one or more pharmaceutically acceptable excipients as finely divided powders. Pharmaceutically acceptable excipients particularly suited for use in dry powders are known to those skilled in the art and include lactose, starch, mannitol, and mono-, di-, and polysaccharides.

The dry powder compositions for use in accordance with the present invention are administered via inhalation devices. As an example, such devices can encompass capsules and cartridges of for example gelatin, or blisters of, for example, laminated aluminum foil. In various embodiments, each capsule, cartridge or blister may contain doses of composition according to the teachings presented herein. Examples of inhalation devices can include those intended for unit dose or multi-dose delivery of composition, including all of the devices set forth herein. As an example, in the case of multi-dose delivery, the formulation can be pre-metered (e.g., as in Diskus®, see GB2242134, U.S. Pat. Nos. 6,032,666, 5,860,419, 5,873,360, 5,590,645, 6,378,519 and 6,536,427 or Diskhaler, see GB 2178965, 2129691 and 2169265, U.S. Pat. Nos. 4,778,054, 4,811,731, 5,035,237) or metered in use (e.g., as in Turbuhaler, see EP 69715, or in the devices described in U.S. Pat. No. 6,321,747). An example of a unit-dose device is Rotahaler (see GB 2064336). In one embodiment, the Diskus® inhalation device comprises an elongate strip formed from a base sheet having a plurality of recesses spaced along its length and a lid sheet peelably sealed thereto to define a plurality of containers, each container having therein an inhalable formulation containing the compound optionally with other excipients and additive taught herein. The peelable seal is an engineered seal, and in one embodiment the engineered seal is a hermetic seal. Preferably, the strip is sufficiently flexible to be wound into a roll. The lid sheet and base sheet will preferably have leading end portions which are not sealed to one another and at least one of the leading end portions is constructed to be attached to a winding means. Also, preferably the engineered seal between the base and lid sheets extends over their whole width. The lid sheet may preferably be peeled from the base sheet in a longitudinal direction from a first end of the base sheet.

A dry powder composition may also be presented in an inhalation device which permits separate containment of two different components of the composition. Thus, for example, these components are administrable simultaneously but are stored separately, e.g., in separate pharmaceutical compositions, for example as described in WO 03/061743 A1 WO 2007/012871 A1 and/or WO2007/068896, as well as U.S. Pat. Nos. 8,113,199, 8,161,968, 8,511,304, 8,534,281, 8,746,242 and 9,333,310.

In one embodiment, an inhalation device permitting separate containment of components is an inhaler device having two peelable blister strips, each strip containing pre-metered doses in blister pockets arranged along its length, e.g., multiple containers within each blister strip, e.g., as found in ELLIPTA®. Said device has an internal indexing mechanism which, each time the device is actuated, peels open a pocket of each strip and positions the blisters so that each newly exposed dose of each strip is adjacent to the manifold which communicates with the mouthpiece of the device. When the patient inhales at the mouthpiece, each dose is simultaneously drawn out of its associated pocket into the manifold and entrained via the mouthpiece into the patient's respiratory tract. A further device that permits separate containment of different components is DUGHALER™ of Innovata. In addition, various structures of inhalation devices provide for the sequential or separate delivery of the pharmaceutical composition(s) from the device, in addition to simultaneous delivery.

Aerosols may be formed by suspending or dissolving Compound I, or a pharmaceutically acceptable salt thereof, in a liquefied propellant. Suitable propellants include halocarbons, hydrocarbons, and other liquefied gases. Representative propellants include: trichlorofluoromethane (propellant 11), dichlorofluoromethane (propellant 12), dichlorotetrafluoroethane (propellant 114), tetrafluoroethane (HFA-134a), 1,1-difluoroethane (HFA-152a), difluoromethane (HFA-32), pentafluoroethane (HFA-12), heptafluoropropane (HFA-227a), perfluoropropane, perfluorobutane, perfluoropentane, butane, isobutane, and pentane. Aerosols comprising Compound I, or a pharmaceutically acceptable salt thereof, will typically be administered to a patient via a metered dose inhaler (MDI). Such devices are known to those skilled in the art.

The aerosol may contain additional pharmaceutically acceptable excipients typically used with multiple dose inhalers such as surfactants, lubricants, co-solvents and other excipients to improve the physical stability of the formulation, to improve valve performance, to improve solubility, or to improve taste.

Suspensions and solutions comprising Compound I, or a pharmaceutically acceptable salt thereof, may also be administered to a patient via a nebulizer. The solvent or suspension agent utilized for nebulization may be any pharmaceutically acceptable liquid such as water, aqueous saline, alcohols or glycols, e.g., ethanol, isopropyl alcohol, glycerol, propylene glycol, polyethylene glycol, etc. or mixtures thereof. Saline solutions utilize salts which display little or no pharmacological activity after administration. Both organic salts, such as alkali metal or ammonium halogen salts, e.g., sodium chloride, potassium chloride or organic salts, such as potassium, sodium and ammonium salts or organic acids, e.g., ascorbic acid, citric acid, acetic acid, tartaric acid, etc. may be used for this purpose.

Other pharmaceutically acceptable excipients may be added to the suspension or solution. Compound I, or a pharmaceutically acceptable salt thereof, may be stabilized by the addition of an inorganic acid, e.g., hydrochloric acid, nitric acid, sulfuric acid and/or phosphoric acid; an organic acid, e.g., ascorbic acid, citric acid, acetic acid, and tartaric acid, etc., a complexing agent such as EDTA or citric acid and salts thereof; or an antioxidant such as antioxidant such as vitamin E or ascorbic acid. These may be used alone or together to stabilize Compound I, or a pharmaceutically acceptable salt thereof. Preservatives may be added such as benzalkonium chloride or benzoic acid and salts thereof. Surfactant may be added particularly to improve the physical stability of suspensions. These include lecithin, disodium dioctylsulphosuccinate, oleic acid and sorbitan esters.

One embodiment of the invention encompasses combinations comprising one or two other therapeutic agents. It will be clear to a person skilled in the art that, where appropriate, the other therapeutic ingredient(s) may be used in the form of salts, for example as alkali metal or amine salts or as acid addition salts, or prodrugs, or as esters, for example lower alkyl esters, or as solvates, for example hydrates to optimize the activity and/or stability and/or physical characteristics, such as solubility, of the therapeutic ingredient. It will be clear also that, where appropriate, the therapeutic ingredients may be used in optically pure form.

The combinations referred to above may conveniently be presented for use in the form of a pharmaceutical formulation and thus pharmaceutical formulations comprising a combination as defined above together with a pharmaceutically acceptable diluent or carrier represent a further aspect of the invention. Artigas, A, et al., Inhalation therapies in acute respiratory distress syndrome, Ann Transl Med. 2017 July; 5(14):293. doi: 10.21037/atm.2017.07.21. Review

The individual compounds of such combinations may be administered either sequentially or simultaneously in separate or combined pharmaceutical formulations. In one embodiment, the individual compounds will be administered simultaneously in a combined pharmaceutical formulation. Appropriate doses of known therapeutic agents will readily be appreciated by those skilled in the art.

The invention thus provides, in a further aspect, a pharmaceutical composition comprising a combination of Compound I, or a pharmaceutically acceptable salt thereof, together with another therapeutically active agent.

Suitably, for the treatment of ALI, ARDS and MODS, Compound I, or a pharmaceutically acceptable salt thereof, or pharmaceutical formulations of the invention may be administered together with an anti-inflammatory agent such as, for example, a corticosteroid, or a pharmaceutical formulation thereof. For example, Compound I, or a pharmaceutically acceptable salt thereof, may be formulated together with an anti-inflammatory agent, such as a corticosteroid, in a single formulation, such as a dry powder formulation for inhalation. Alternatively, a pharmaceutical formulation comprising Compound I, or a pharmaceutically acceptable salt thereof, may be administered in conjunction with a pharmaceutical formulation comprising an anti-inflammatory agent, such as a corticosteroid, either simultaneously or sequentially. In one embodiment, a pharmaceutical formulation comprising Compound I, or a pharmaceutically acceptable salt thereof, and a pharmaceutical formulation comprising an anti-inflammatory agent, such as a corticosteroid, may each be held in device suitable for the simultaneous administration of both formulations via inhalation.

Suitable corticosteroids for administration together with Compound I, or a pharmaceutically acceptable salt thereof, include, but are not limited to, fluticasone furoate, fluticasone propionate, beclomethasone dipropionate, budesonide, ciclesonide, mometasone furoate, triamcinolone, flunisolide and prednisolone. In one embodiment of the invention a corticosteroid for administration together with Compound I, or a pharmaceutically acceptable salt thereof, via inhalation includes fluticasone furoate, fluticasone propionate, beclomethasone dipropionate, budesonide, ciclesonide, mometasone furoate, and, flunisolide.

Suitably, compounds or pharmaceutical formulations of the invention may be administered together with one or more bronchodilators, or pharmaceutical formulations thereof. For example, Compound I, or a pharmaceutically acceptable salt thereof, may be formulated together with one or more bronchodilators in a single formulation, such as a dry powder formulation for inhalation. Alternatively, a pharmaceutical formulation comprising Compound I, or a pharmaceutically acceptable salt thereof, may be administered in conjunction with a pharmaceutical formulation comprising one or more bronchodilators, either simultaneously or sequentially. In a further alternative, a formulation comprising Compound I, or a pharmaceutically acceptable salt thereof, and a bronchodilator may be administered in conjunction with a pharmaceutical formulation comprising a further bronchodilator. In one embodiment, a pharmaceutical formulation comprising Compound I, or a pharmaceutically acceptable salt thereof, and a pharmaceutical formulation comprising one or more bronchodilators may each be held in device suitable for the simultaneous administration of both formulations via inhalation. In a further embodiment, a pharmaceutical formulation comprising Compound I, or a pharmaceutically acceptable salt thereof, together with a bronchodilator and a pharmaceutical formulation comprising a further bronchodilator may each be held in device suitable for the simultaneous administration of both formulations via inhalation.

Suitable bronchodilators for administration together with Compound I, or a pharmaceutically acceptable salt thereof, include, but are not limited to, β₂-adrenoreceptor agonists and anticholinergic agents. Examples of β₂-adrenoreceptor agonists, include, for example, vilanterol, salmeterol, salbutamol, formoterol, salmefamol, fenoterol carmoterol, etanterol, naminterol, clenbuterol, pirbuterol, flerbuterol, reproterol, bambuterol, indacaterol, terbutaline and salts thereof, for example the xinafoate (1-hydroxy-2-naphthalenecarboxylate) salt of salmeterol, the sulphate salt of salbutamol or the fumarate salt of formoterol. Suitable anticholinergic agents include umeclidinium (for example, as the bromide), ipratropium (for example, as the bromide), oxitropium (for example, as the bromide) and tiotropium (for example, as the bromide). In one embodiment of the invention, Compound I, or a pharmaceutically acceptable salt thereof, may be administered together with a β₂-adrenoreceptor agonist, such as vilanterol, and an anticholinergic agent, such as, umeclidinium.

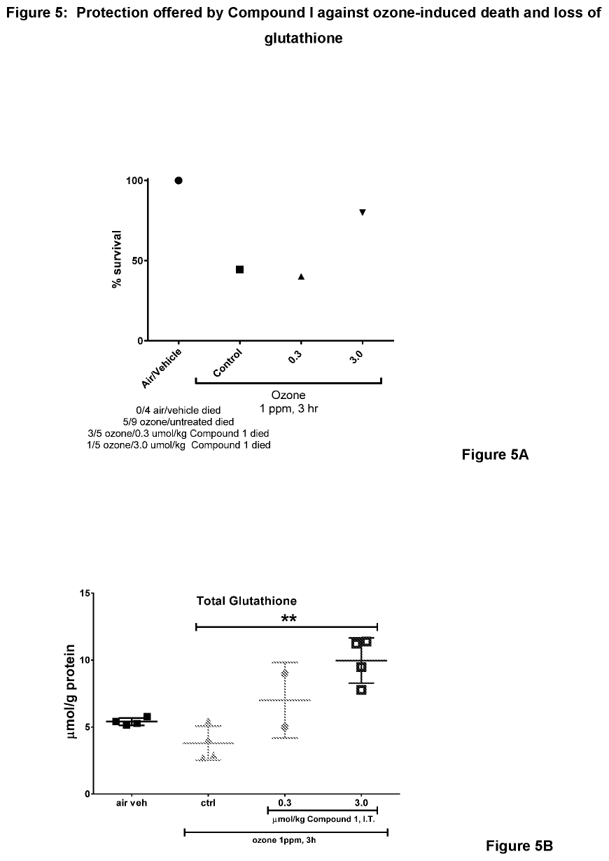

The model of PQ-induced increase in lung edema formation described in Rumsey, W., et al., (Mutagenesis. 2017, 32(3):343-353) is incorporated by reference in its entirety. In support of this invention, ALI was stimulated by the tracheal instillation of PQ which damages mtDNA in lung cells and provokes a loss of epithelial/endothelial integrity and edema formation. The data below show that Compound I improves lung function, while protecting against the PQ-induced increase in lung edema formation, i.e., wet:dry wt. ratio. Moreover, the PQ-mediated mtDNA damage is also prevented by drug treatment. In support of the latter findings, Compound I stimulated an upregulation of NQO1 activity while attenuating the impact of PQ on DNA oxidation. Using another form of lung injury, i.e., ozone inhalation, edema formation and mtDNA damage are protected and in a more severe case of ozone exposure the drug treated animals survive the lethal insult. See FIG. 5.

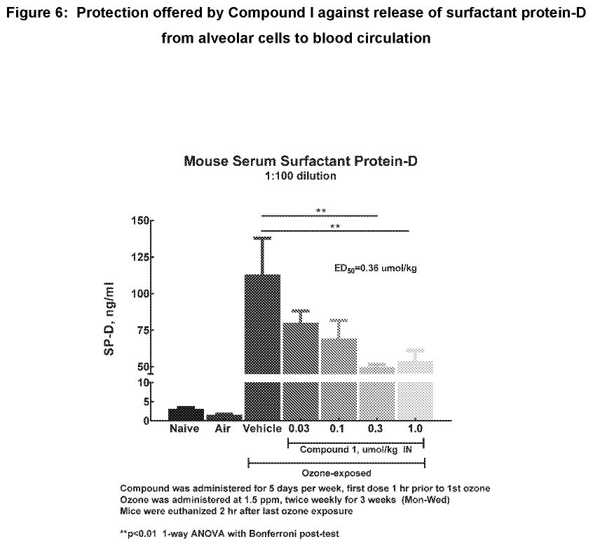

In another case (See FIG. 6) using repeated ozone exposures, changes in mtDNA and surfactant D, a biomarker of the integrity of the epithelial/alveolar barrier, were dose dependently prevented by Compound I treatment.

### Methods

Age-matched male Lewis rats (250-400 g, Charles River Breeding Laboratories, Wilmington, Mass.) were allowed free access to food and water. Animals were administered NRF2 compound via tracheal instillation 24 hours prior to oxidative insult.

For studies using PQ (or N,N′-dimethyl-4,4′-dihydrochloride, Sigma Aldrich, St Louis, Mo.) as the toxin, aliquots were prepared in sterile phosphate-buffered saline (PBS) and instilled directly to the trachea of the rat while under isoflurane anesthesia. Rats were anesthetized in a small acrylic induction box with 2-5% isoflurane gas. When surgical anesthesia was obtained (assessed by loss of the righting reflex followed by use of the pedal withdrawal reflex), the rat was removed from the box and placed supine on a head up, tilted platform. The trachea was illuminated and either PQ or vehicle (300 μl, doses identified in figure legends) was directly instilled into the trachea, anterior to the primary bifurcation at the carina, using a blunt-tipped needle. The animal was returned to a recovery cage, where the righting reflex was regained in 2-3 minutes.

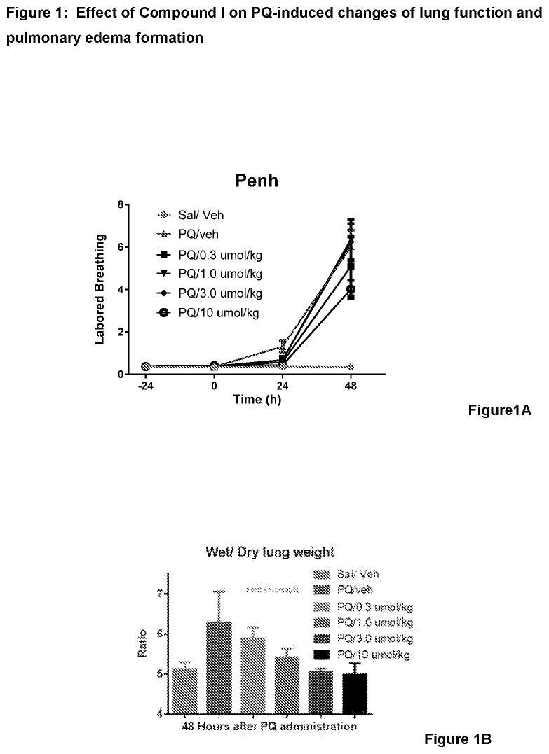

To monitor changes in airway mechanics, rats were placed into individual plethysmograph chambers (BUXCO Electronics, Troy, N.Y.). Fresh air was supplied by bias flow pumps to the chambers. Baseline respiratory (Penh) values were collected prior to administration of PQ and on succeeding days after administration of the agent. An average Penh was calculated for a period of 5 min where enhanced pause (Penh=[(expiratory time/relaxation time)−1]×(peak expiratory flow/peak inspiratory flow)) and relaxation time is the amount of time required for 70% of the tidal volume to be expired. See FIG. 1A.

For measurements of edema formation in the lungs, the tissue was excised and weighed gravimetrically. A portion was dried overnight in an oven at 60 degrees F. and weighed for dry weight. See FIG. 1B.

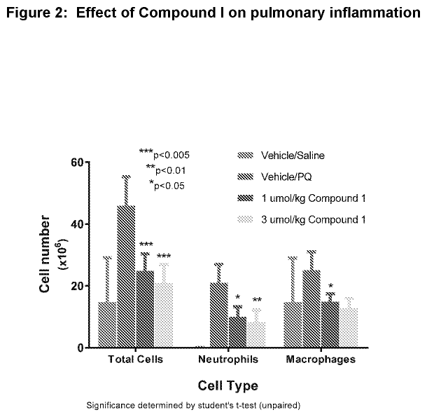

In some studies, bronchoalveolar lavage was performed to identify the immune cells infiltrating the lung in response to the toxicant. After the animals were euthanized (Fatal Plus, 100 mg/kg i.p.), the trachea was surgically exposed and a blunt-tipped needle was inserted into the trachea for administration of lavage fluid (5×5 ml Dulbecco's phosphate buffered saline, PBS). The lavage fluid was collected, placed on ice and centrifuged (3000 rpm×10 min, Beckman-Coulter, Danvers, Mass.). Supernatant was aspirated and frozen whereas the pellet was resuspended in 5 ml of PBS. An aliquot (100 μl) was centrifuged (300 rpm×5 min, cytospin, Thermo-Shandon, Waltham, Mass.) and a separate sample prepared as 1:5 dilution for total cell counts using a hemocytometer. Aliquots of cells were placed on slides and stained (Kwik-Diff-Quick, Thermo-Shandon, Waltham, Mass.) according to manufacturer's instructions. At least 200 cells were counted and percentages of different cell types were calculated (macrophages and neutrophils). See FIG. 2.

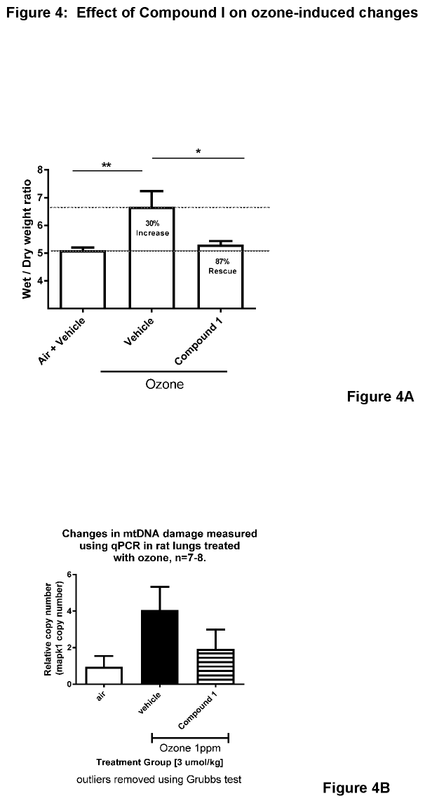

In another study, rats were exposed to ozone (2.0 ppm) for a 3 hr period twice a week with a resting period of 2 days between treatments. In some cases, ozone (1 ppm) was applied twice per week for three consecutive weeks. Ozone was generated (Oxycycler ozonator (model# A84ZV, Biospherix Inc., Lacona, N.Y.) by passing room air through the ozonator at a rate of 50-75 cm³/min, mixing it with filtered room air at a rate of 10 L/min, and flowing this sample into a Plexiglass chamber containing the rodents. Ozone, carbon dioxide and humidity levels in the chamber were constantly monitored (Ozone Monitor Model 450, Teledyne Advanced Pollution Instrumentation, Inc., Thousand Oaks, Calif.). The animals were euthanized as described above 24 hrs after the last exposure to ozone. See FIG. 4-5.

Total DNA (or RNA) was extracted from samples of the frozen right inferior pulmonary lobe excised from animals exposed to toxicant or from respective sham animals. For extraction of either RNA or DNA, the tissue was added to lysis buffer (Kingfisher DNA or RNA extraction kit, Thermo Fisher Scientific, Waltham, Mass.) and the samples were processed according to the manufacturer's instructions. The nucleotide quantities were determined with respective Qubit kits (Thermo Fisher, Waltham, Mass.). For measurement of mtDNA copy number by quantitative real time PCR, nuclear and mitochondrial primer sets (1 pmol/μl), and 2× SYBR green master mix (Life Technologies, Waltham, Mass.) were added to 50 ng of DNA combined with water (total 20 ul). The reaction was run according to the following protocol: 95° C.×20 sec, then 40 cycles of 95° C.×1 sec and 60° C.×20 sec followed by a melt curve of one cycle of 95° C.×15 sec, 60° C.×60 sec, and 95° C.×15 sec (Viia 7, Life Technologies, Waltham, Mass., and Viia 7 software version 1.2.2). The primer sequences were:

Primer 2 sense:
(SEQ ID NO: 1)
CTCTCACCCTATTAACCACT,
Primer 2 antisense:
(SEQ ID NO: 2)
GTTAAAAGTGCATACCGCCA,
MAPK1 sense:
(SEQ ID NO: 3)
GCTTATGATAATCTCAACAAAGTTCG,
and
MAPK1 antisense:
(SEQ ID NO: 4)
ATGTTCTCATGTCTGAAGCG
for the mitochondrial and
nuclear primer sets respectively.

Relative copy number was calculated using the modified delta CT method as previously described (20) and expressed as a relative fold-change based upon control values with confidence intervals.

For determination of mtDNA damage (21), 15 ng of DNA in long chain PCR buffer was coupled with appropriate long and short primers for murine tissues. The reaction mixture was essentially the same for both long and short runs with the exception that the [Mg++] was 1.2 and 1.1 mM, respectively. For mouse tissues, the following thermocycler conditions were utilized: 94° C.×2 min followed by 19 cycles at 94° C.×15 sec, 64° C.×30 sec, 68° C.×8 min and finished at 72° C.×7 min; 94° C.×2 min, 94° C.×15 sec, 60° C.×30 sec, 72° C.×45 sec, and finished at 72° C.×7 min for the long and short PCR, respectively. The long and short primer sequences for mouse;

10 kb mitochondrial sense
(SEQ ID NO: 5)
5′-GCCAGCCTGACCCATAGCCATAATAT-3′,
10 kb mitochondrial antisense
(SEQ ID NO: 6)
5′-GAGAGATTTTATGGGTGTAATGCGG-3′,
117 bp fragment sense
(SEQ ID NO: 7)
5′-CCCAGCTACTACCATCATTCAAGT-3′,
and
117 bp fragment antisense
(SEQ ID NO: 8)
5′-GATGGTTTGGGAGATTGGTTGATGT-3′.

For samples obtained from rat lungs, the procedures were similar with the following exceptions: the thermocycler reaction conditions for long PCR were: 2 min incubation at 94° C. followed by 20 cycles at 94° C.×15 sec, 65° C.×30 sec, and 68° C.×8 min, and then finished at 72° C.×7 min and 94° C.×2 min. The short reaction was carried out as for the mouse. The long and short primer sequences for rat were:

(SEQ ID NO: 9)
5′-AAAATCCCCGCAAACAATGACCACCC-3′,
(SEQ ID NO: 10)
5′ GGCAATTAAGAGTGGGATGGAGCCAA-3′,
(SEQ ID NO: 11)
5′-CCTCCCATTCATTATCGCCGCCCTTGC-3′,
and
(SEQ ID NO: 12)
5′-GTCTGG GTCTCCTAGTAGGTCTGGGAA-3′.

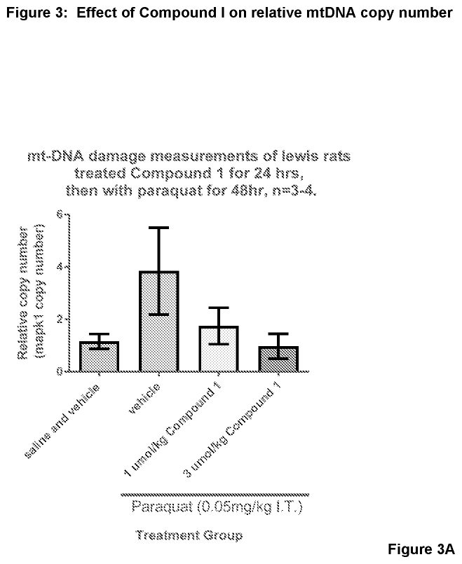

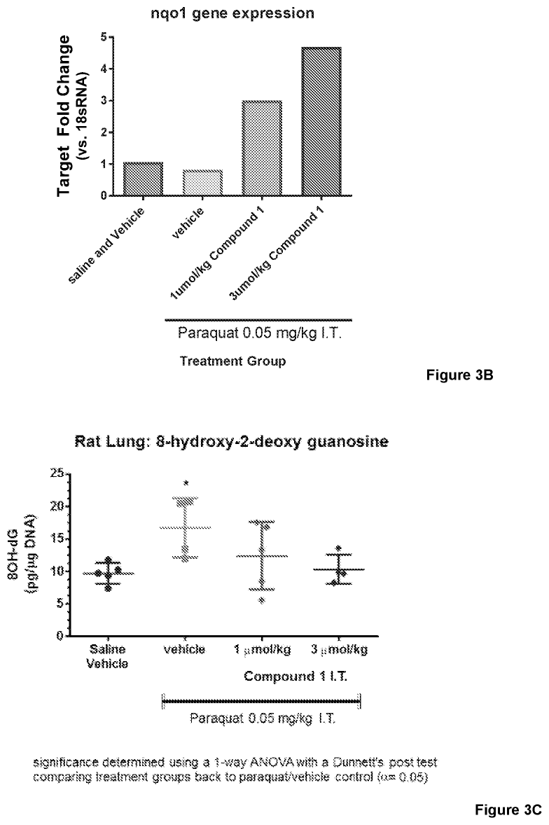

For both species, the long and short PCR products were then diluted 1:10 with Tris EDTA buffer containing 5 μl/ml of Pico Green (Molecular Probes, Invitrogen, Carlsbad Calif.) and fluorescence was monitored (485 nm excitation/528 nm emission, Envision Perkin Elmer, Waltham, Mass.). The replicates for each sample were averaged, the long primer was subtracted from the short primer, and transformed into percent of control using normalization functions (GraphPad Prism v6.0, La Jolla, Calif.). The data were calculated to reflect an increase in damage by subtracting the long primer from the short primer, rather than the opposite which would show the reduction of signal. See FIG. 3.

Surfactant protein-D (SP-D) was measured using a commercially available kit (Mouse Quantikine SP-D, R&D Systems #MSFPDO, Minneapolis, Minn.). Absorbances were monitored at 450 and 540 nm using a microplate spectrophotometer (Powerwave, BioTek, Winooski, Vt.) with Gen5 software (version 2.03.Ink). Blood was collected via cardiac puncture from mice anesthetized with 3-5% isoflurane, allowed to congeal at room temperature for 30 min, and centrifuged (3000 rpm×10 min). The serum was removed and placed into 96 well microplates (Nunc Maxisorp microplates, #12-565-135, Thermoscientific, Rochester, N.Y.) at −20° C. until assayed. Degradation or breakdown of alveolar barrier and leakage of water into the alveolar is measured by wet:dry ratio. With the degradation of the barrier there is movement of surfactant-D into the circulation, which is a biomarker of COPD. By administering Compound I, the degradation of the alveolar barrier is prevented. See FIG. 6.

The above description fully discloses the invention including preferred embodiments thereof. Modifications and improvements of the embodiments specifically disclosed herein are within the scope of the following claims. Without further elaboration, it is believed that one skilled in the art can, using the preceding description, utilize the present invention to its fullest extent. Therefore, the Examples herein are to be construed as merely illustrative and not a limitation of the scope of the present invention in any way. The embodiments of the invention in which an exclusive property or privilege is claimed are defined as follows.

1. A method for treating acute lung injury in a mammal in need thereof, comprising administering an effective amount of an NRF2 activator to said mammal, wherein the NRF2 activator is (S)-3-(3-(((R)-4-ethyl-1,1-dioxido-3,4-dihydro-2H-pyrido[2,3-b][1,4,5]oxathiazepin-2-yl)methyl)-4-methylphenyl)-3-(1-ethyl-4-methyl-1H-benzo[d][1,2,3]triazol-5-yl)propanoic acid, or a pharmaceutically acceptable salt thereof.

2. A method for treating acute respiratory distress syndrome in a mammal in need thereof, comprising administering an effective amount of an NRF2 activator to said mammal, wherein the NRF2 activator is (S)-3-(3-(((R)-4-ethyl-1,1-dioxido-3,4-dihydro-2H-pyrido[2,3-b][1,4,5]oxathiazepin-2-yl)methyl)-4-methylphenyl)-3-(1-ethyl-4-methyl-1H-benzo[d][1,2,3]triazol-5-yl)propanoic acid, or a pharmaceutically acceptable salt thereof.

3. A method for treating multiple organ dysfunction syndrome in a mammal in need thereof, comprising administering an effective amount of an NRF2 activator to said mammal, wherein the NRF2 activator is (S)-3-(3-(((R)-4-ethyl-1,1-dioxido-3,4-dihydro-2H-pyrido[2,3-b][1,4,5]oxathiazepin-2-yl)methyl)-4-methylphenyl)-3-(1-ethyl-4-methyl-1H-benzo[d][1,2,3]triazol-5-yl)propanoic acid, or a pharmaceutically acceptable salt thereof.

4. A method of treating the symptoms of acute lung injury in a mammal in need thereof, comprising administering an effective amount of an NRF2 activator to said mammal, wherein the NRF2 activator is (S)-3-(3-(((R)-4-ethyl-1,1-dioxido-3,4-dihydro-2H-pyrido[2,3-b][1,4,5]oxathiazepin-2-yl)methyl)-4-methylphenyl)-3-(1-ethyl-4-methyl-1H-benzo[d][1,2,3]triazol-5-yl)propanoic acid, or a pharmaceutically acceptable salt thereof.

5. The method of claim 4 wherein the symptoms include an accumulation of alveolar fluid, hypoxemia, cough, wheezing, dyspnea, hyperpnea and pulmonary inflammation.

6. The method as claimed in claim 1, wherein the NRF2 activator is (S)-3-(3-(((R)-4-ethyl-1,1-dioxido-3,4-dihydro-2H-pyrido[2,3-b][1,4,5]oxathiazepin-2-yl)methyl)-4-methylphenyl)-3-(1-ethyl-4-methyl-1H-benzo[d][1,2,3]triazol-5-yl)propanoic acid.

7-18. (canceled)

19. The method as claimed in claim 2, wherein the NRF2 activator is (S)-3-(3-(((R)-4-ethyl-1,1-dioxido-3,4-dihydro-2H-pyrido[2,3-b][1,4,5]oxathiazepin-2-yl)methyl)-4-methylphenyl)-3-(1-ethyl-4-methyl-1H-benzo[d][1,2,3]triazol-5-yl)propanoic acid.

20. The method as claimed in claim 3, wherein the NRF2 activator is (S)-3-(3-(((R)-4-ethyl-1,1-dioxido-3,4-dihydro-2H-pyrido[2,3-b][1,4,5]oxathiazepin-2-yl)methyl)-4-methylphenyl)-3-(1-ethyl-4-methyl-1H-benzo[d][1,2,3]triazol-5-yl)propanoic acid.

21. The method as claimed in claim 4, wherein the NRF2 activator is (S)-3-(3-(((R)-4-ethyl-1,1-dioxido-3,4-dihydro-2H-pyrido[2,3-b][1,4,5]oxathiazepin-2-yl)methyl)-4-methylphenyl)-3-(1-ethyl-4-methyl-1H-benzo[d][1,2,3]triazol-5-yl)propanoic acid.

22. The method as claimed in claim 5, wherein the NRF2 activator is (S)-3-(3-(((R)-4-ethyl-1,1-dioxido-3,4-dihydro-2H-pyrido[2,3-b][1,4,5]oxathiazepin-2-yl)methyl)-4-methylphenyl)-3-(1-ethyl-4-methyl-1H-benzo[d][1,2,3]triazol-5-yl)propanoic acid.
